(19) United States
(12) Patent Application Publication
(10) Pub. No.: US 2006/0260609 A1
(43) Pub. Date: Nov. 23, 2006

(54) Therapy device for the specifically local treatment of illnesses transmitted by droplet infection, in particular colds

(76) Inventor: Johann Bruening, Berlin (DE)

Correspondence Address: D. PETER HOCHBERG CO. L.P.A., 1940 EAST 6TH STREET, CLEVELAND, OH, 44114, US

(21) Appl. No.: 11/201,777

(22) Filed: Aug. 11, 2005

(30) Foreign Application Priority Data
May 20, 2005 (DE) 102 005 023 463.1

(51) Int. Cl. A62B 9/00; A61M 15/00

(52) U.S. Cl. 128/200.24

(57) ABSTRACT

A therapy device for specifically local short-term treatment of infectious illnesses transmitted by droplet infection, such as colds, by a heated dry current of air which is warmed up and acts directly and in a locally restricted manner onto the epithelium of the nasal cavities and the adjacent areas in the nasal/throat region. The therapy device profits of the knowledge that it is easier to combat symptoms caused by colds, when the dry heated air current which is used has a relatively high temperature in the range of noticeably above 50° C.

### BACKGROUND OF THE INVENTION

1. Field of the Invention

The invention concerns a device for the treatment of symptoms and illnesses caused by colds in humans, more particularly within the nasal cavities, the frontal sinuses and the throat region as well as all other infectious illnesses which are transmitted by droplet infection and penetrate the body through the nasal/throat region.

2. Description of the Prior Art

International Publication WO 03/097143 A1 describes in detail the state of virological research concerning common colds and their causes including the prior art devices employed for more or less effective therapeutical measures. The easy to handle table device for producing an air current between a cold air inlet side and a warm air discharge side for local treatment of colds by an ionised heated current of air which is warmed up at least to the human central temperature and acts directly and in a locally restricted manner, as described in the WO document, has proved successful in practice for curing or at least easing colds which are commonly or specifically called “a runny nose”. The therapy approach to colds and all other infectuous illnesses transmitted by droplet infection by using the known device resides, on the one hand, in the knowledge that a specifically local treatment by air heated above the human central temperature has a favorable effect on warding off such symptoms, in particular if, on the other hand, the warmed air current prior to its emergence from the housing is passed over a crystal salt charge for negative ionization thereof. The cited document already assumes that rhinoviruses can be combatted more successfully the higher the air temperatures used for the treatment are.

Although there has thus been the assumption that higher application temperatures could promote the therapeutical effect when treating colds, fundamentally there were objections as to the specific increase of this temperature noticeably above 50° C. However, it has been found out in the meantime by numerous tests that treatment temperatures above 50° C. can be utilized for a specifically local treatment of the epithelium of the nasal cavities and the adjacent areas in the nasal/throat area, provided dry air, if possible very dry air is employed, the word “dry” meaning a relative air moisture of less than 35%, preferably of less than 20%.

### SUMMARY OF THE PRESENT INVENTION

Based on this knowledge, the invention proposes a therapy device for a specifically local short-term treatment of colds by a heated air current which acts directly and in a locally restricted manner onto the epithelium of the nasal cavities and the adjacent areas in the nasal/throat area, the therapy device being designed as an easy to handle device housing including a device for producing an air current between a cold air intake side and a hot air discharge side on the one hand, and a controllable heating device for heating the air current to an outlet temperature of at least 50° C., on the other hand, being integrated therein.

It is advantageous if the device producing the current of air and the controllable heating device are matched so that the exit temperature of the heated dry air current is in a range of from 50° C. to 120° C., preferably in a range of from 55° C. to 85° C., and in particular at about 70° C.

The specific short-term treatment of colds and all other infectuous illnesses transmitted by droplet infection with hot dry air primarly through the nasal cavities is ensured by a settable timer for presetting the switch-off time of the device which, for example, can be set in a range of from one minute to six minutes of treatment in steps of one minute. This timer can be coupled to an air temperature and heating power regulator for presetting temperature changes of the mentioned air current in a time-dependent manner.

Advantageously, an air volume throughput regulator is associated with the heating power regulator for stepwise changing the air exit temperature according to the steps of the timer.

The therapy device under the present invention is advantageously designed as a table device having a device housing composed of a plurality of housing parts, for example, two or three housing parts, together with a single-piece base part for setting the table device down on a support surface. The base part comprises an air current producing apparatus and the controllable heating device which can be automatically switched, for example, in steps. A removeable hollow clip-on part having an upper hot air discharge opening fittingly joins the base on the upper side. This clip-on part is bevelled in the region of the air discharge opening with respect to the vertical and has a rounded soft edge matched to the mouth/nasal area of the user of the device.

The controllable heating device may be designed as a stepwise switchable PTC element.

Measures for preventing electromagnetic radiation of a higher frequency from penetrating outwards are taken in the power supply device, the electronic unit for controlling the heating device and when driving the air current producing device and the heating device.

Also with the therapy device presented here for the specifically local short-term treatment of colds and all other infectious illnesses transmitted by droplet infection by very hot dry air can be passed over a charge of crystal salt removeably held in the device housing, for example, in the form of a salt tablet or a salt pad, as described in WO 03/097143 A1, for preferably negative ionization of the heated air current prior to its emergence from the housing. Usually, it may be assumed that the therapy device according to the invention is operated in the domestic domain, namely in a bathroom or living room, at normal ambient temperatures and a correspondingly average relative air moisture content. Owing to the relatively high temperature of the air current, not only the relative air moisture at the air outlet is much lower, but additionally the air current is dried by the heating device. With a relatively high air moisture, it may be advantageous, unless a lower treatment temperature is selected from the outset, to predry the sucked-in air in that a dehumidifying bed provided on the intake side of the therapy device in the interior of the housing or designed as a separate connecting unit is allowed to be passed through by the heated air current, said dehumidifying bed containing, for example, silica gel.

### BRIEF DESCRIPTION OF THE DRAWINGS

The invention and advantageous details thereof are explained below with reference to the drawing in exemplary and well-tested embodiments corresponding to the presently best design choice. These show:

FIG. 1 is a schematic sectional side view of the therapy device with features according to the invention, and

FIG. 2 is an isometric representation of a partial section of a therapy device according to the invention.

### DETAILED DESCRIPTION OF THE PRESENT INVENTION

Like reference characters refer to like parts or constructional groups.

As can be seen from the two figures of the drawings, the illustrated therapy device according to the invention for efficiently treating colds and other infectuous illnesses transmitted by droplet infection, namely for local short-term treatment by heated dry air is designed as a table device with an inclination of about 10° to 35° with respect to the vertical. According to the illustrated embodiment, the device 1 comprises three matched housing parts, namely a housing base part 4 to which three supporting legs 12 are attached on the bottom side providing a slip-proof firm stand on a support surface, for example, a table, and sufficient ground clearance on the underside of the device, thereby ensuring free inflow of air, an inner housing part 3 integrally connected to the base part 4 on the bottom side and substantially comprising an upwardly tapering tube holding in its lower widened portion an electric motor 8 on the axis of which is mounted a ventilating fan 7 driven thereby. The axes of the motor 8 and the ventilating fan 7, respectively, lie on the inclined axis of the inner housing part 3. The third housing part is formed by an upper external housing part 2 encompassing the housing part 3 more or less tightly in the upper region, then changing to a bulged region and forming a covering console 22 at the operating or front end of the device. The lower circumferential edge of the upper housing part 2 including the lower edge of the covering console 22 encompasses an upper circumferential edge of the lower housing part 4. The connection between the upper housing part 2 and the lower housing part 4 can be realized by clamp fit, plastic screws and/or bonding. A hollow-conical clip-on part 16 is slipped onto the upper free end of the upper housing part 2 in an accurate fit and a positive form-fitting manner, which clip-on part has on its upper side a termination bevelled in the direction of the operating console with a rounded circumferential border 17 whose inner contour is adapted to the nasal/mouth area of a human face. Mounted in the upper tubular region of the inner housing part 3 above the ventilating fan 7, there is an annular assembly of electric heating elements 6 (ASM elements or the like), allowing preselectable heating to a high temperature of the air sucked in by the ventilating fan 7 from below, when the device is in operation. The upper circumferential inner edge of the upper housing part 3 may be designed as a clamping support ring for a compressed tablet or pill 20 of crystal salt which can be exchanged on removal of the clip-on part 16. The advantageous therapeutical effect of such an ionizing salt charge provided in the air current between the inflow and discharge side of the heated air is described in all detail in the above-cited Document WO 03/097143 A1, the content of which is incorporated by reference in full.

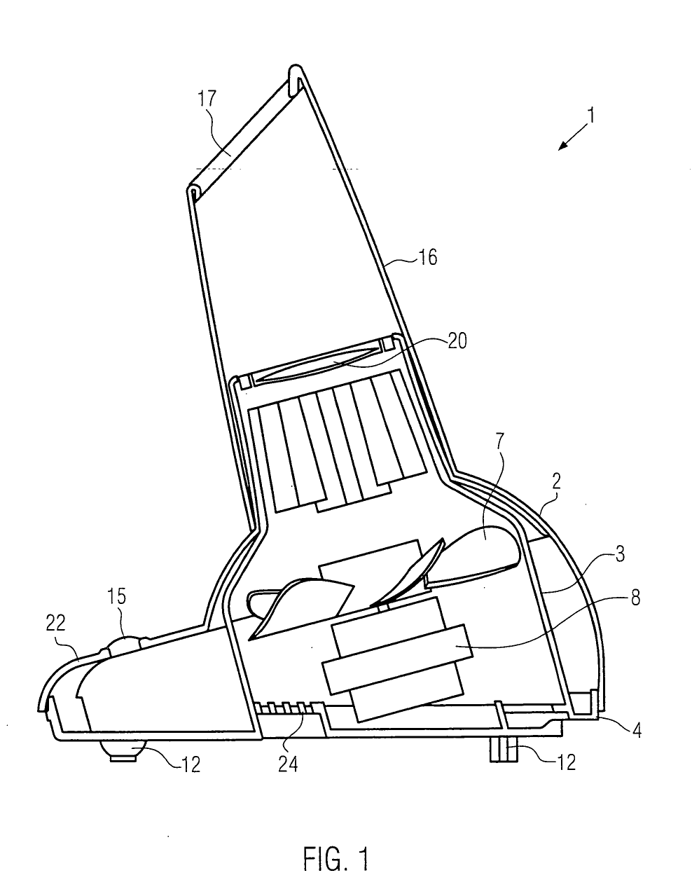

As can be seen from FIG. 1, the base 4 has a circumferential suck-in region 24 projecting inwardly on the bottom side and having ring-shaped circumferential perforations or rows of holes, through which the air to be heated and sucked in by the ventilating 7 on the bottom flows into the interior of the device.

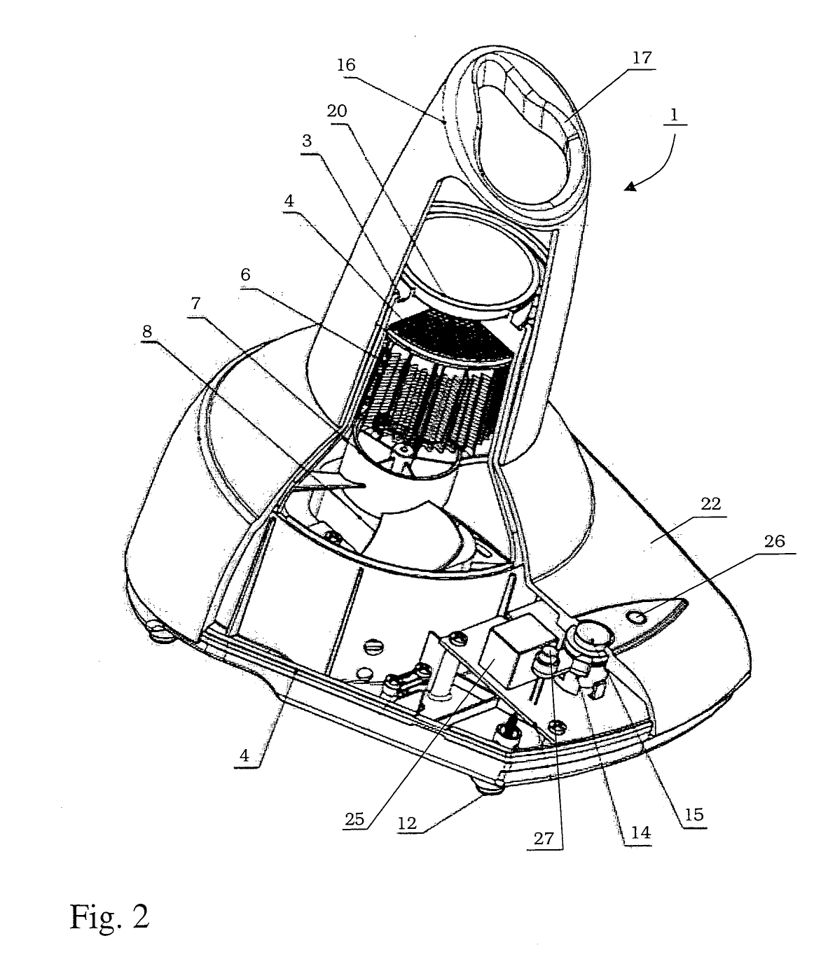

The partly sectional illustration of the operating console 22 in FIG. 2 shows the essential components of the electric operating and control unit of the device. A push button switch 15 is seated on a switch support 14. According to the illustration in FIG. 2, there is a switch-on display 27 on the left hand side and a standby and preset time display 26, preferably different in color, is shown on the right hand side thereof. The power supply unit of the device is not shown. The electronic control unit 25 allows the treatment time to be preferably fixedly preset, for example, between two to eight minutes at a maximum, preferably three minutes. In order to ensure the “foolproof” use of the device, it may be advantageous to fixedly program a maximum operating time of three to four minutes in particular, as recommended by the manufacturer. Also the air volume throughput and the exit temperature, respectively, may be fixedly programmed respectively. However, as a rule, the air exit temperature will be preselectable in steps, for example, via the on/off key 15 springing out in the on-state. Of course it is possible to provide a separate temperature selector in the form of a rotary pushbutton or a rotary wheel at the lower housing edge. In order to prevent overheating of the dry air current exiting at the upper end of the clip-on part 16, there are provided one or several temperature sensors (not shown) at or below the circumferential upper edge of the inner housing part 3.

A small, in particular flatter design of the overall device can be easily implemented, the advantage being that the clip-on part 16 can be made of a flexible tubular material, for example, with a separate clip-on nose/face contour adapter. Also a mains-independent variation of the device has been implemented with exchangeable energy stores, for example, lithium ion batteries. The respective loading device may be integrated either into the appertaining loading cable or directly into the device.

The invention provides an easy to handle therapy device, mainly for the domestic domain, to efficiently reduce or eliminate symptoms of a cold by several short-term treatments, preferably at intervals of one or two hours, for one to six minutes, more preferably for three minutes, by local hyperthermal treatment of the nasal/throat area.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

I claim:

1. A therapy device for the specifically local short-term treatment of infectious illnesses transmitted by droplet infection, such as colds, by a heated dry current of air which is warmed up and acts directly and in a locally restricted manner on the epithelium of the nasal cavities and the adjacent areas in the nasal/throat region, said device comprising an easy to handle device housing, said housing comprising a cold air intake side, a hot air discharge side, a device for producing a current of air between said cold air intake side and said hot air discharge side and a controllable heating device for heating the current of air to an exit temperature of at least 50° C.

2. The therapy device according to claim 1, wherein said device for producing the current of air and said controllable heating device are matched such that the exit temperature of the heated dry current of air is within a temperature range of from 50° C. to 120° C.

3. The therapy device according to claim 2, wherein said air flow and the heating device are matched so that the exit temperature of the heated air current is within a temperature range of from 55° C. to 85° C.

4. The therapy device according to claim 1, wherein said device further comprises a settable timer for presetting a switch-off time of the device.

5. The therapy device according to claim 4, wherein said timer is settable in a range between one minute and six minutes in steps of one minute each.

6. The therapy device according to claim 4, wherein said device further comprises an air temperature and heating power regulator coupled with said timer for presetting temperature changes of the heated current of air in a time-dependent manner.

7. The therapy device according to claim 6, wherein said device further comprises an air volume throughput regulator associated with the heating power regulator for a stepwise change of the air exit temperature, according to the steps of the timer.

8. The therapy device according to claim 1, designed as a table device, wherein said device housing comprises at least one housing part including: a single-piece base part for setting said therapy device down on a support surface, an inner housing part containing an air current production device, a controllable heating device and a removeable hollow-conical clip-on part with an upper heated air discharge opening joined in an accurate fit to the inner housing part.

9. The therapy device according to claim 8, wherein said device further comprises skid-proof legs for supporting said base part at the bottom of said base part to ensure cold air supply on the bottom side, and wherein said housing includes an upper housing part in a front region designed as an operator's console.

10. The therapy device according to claim 1, wherein said controllable heating device comprises at least one PTC element.

11. The therapy device according to claim 10, wherein said device further comprises a temperature sensor on the heated air outlet side for determining performance control of said heating device.

12. The therapy device according to claim 1, wherein said device measures for reducing electric fields (electrosmog) of a higher frequency penetrating outwards.

13. The therapy device according to claim 9, wherein said clip-on part is a flexible tubular clip-on part.

14. The therapy device according to claim 13, wherein said flexible tubular clip-on part is in a two-part form comprising a separate nose/face contour adapter.

15. The therapy device according to claim 1, wherein said device further comprises a mains-independent power supply unit having an energy store integrated into the device housing.

16. The therapy device according to claim 1, wherein said device further comprises a salt charge for negative ionization of the heated air current replaceably positioned in the air current region downstream from the heating device.

17. The therapy device according to claim 3, wherein said air flow and the heating device are matched so that the exit temperature of the heated air current is at a temperature of about 70° C.
